(19) United States
(12) Patent Application Publication
(10) Pub. No.: US 2024/0337475 A1
(43) Pub. Date: Oct. 10, 2024

(54) COIL GEOMETRY FOR AN ELECTROMAGNETIC TRACKING SYSTEM

(71) Applicant: Magnisity Ltd., Hod-HaSharon (IL)

(72) Inventors: Ron BARAK, Tel-Aviv (IL); Benjamin GREENBURG, Hod-HaSharon (IL)

(73) Assignee: Magnisity Ltd., Hod-HaSharon (IL)

(21) Appl. No.: 18/579,404

(22) PCT Filed: Jul. 14, 2022
(86) PCT No.: PCT/IL2022/050762

§ 371 Date: Jan. 15, 2024

Related U.S. Application Data
(60) Provisional application No. 63/222,022, filed on Jul. 15, 2021.

(51) Int. Cl. G01B 7/00; G01B 7/30; G01D 5/20

(52) U.S. Cl. CPC G01B 7/003; G01B 7/30; G01D 5/204

(57) ABSTRACT

A coil topographical geometry including two or more armatures and multiple loops wound about the two or more armatures to form two or more coils. One or more fundamental loops of the multiple loops crosses over themselves at one point in the one or more fundamental loops to form a geometrical shape in the space occupied by the coil topographical geometry. The one or more fundamental loops of the multiple loops are configurable to generate one or more fundamental fields [B_{fun}] that are substantially the same as the fields [Bᵢ(r)] generated by the two or more coils.

### RELATED APPLICATION

This application claims the benefit of priority of U.S. Provisional Patent Application No. 63/222,022 filed on 15 Jul. 2021, the contents of which are incorporated herein by reference in their entirety.

### BACKGROUND

The present disclosure, in some embodiments thereof, relates to Electromagnetic Tracking Systems (EMTS) and, more specifically to coil geometries for a transmitter coil, but not exclusively, to transmitter coils.

Electromagnetic Tracking Systems (EMTS) may be used in various digital fields such as navigation, ballistic tracking, biomechanics, construction, robotics, advanced education and virtual reality and augmented reality systems. For example, in the digital virtual world, the exact position and movements of physical objects or people, enables an avatar to move identically as their original physical characters.

Electromagnetic tracking systems may use 3 orthogonal coils in the receiver to sense the magnetic field in 3 axes using Faraday's Law; the transmitter generates a high-frequency alternating magnetic field (for example, >1 kHz) which creates EMF on the sensing coils. The receiver then amplifies and samples the voltages to recover the derivative of the local magnetic field at its position and orientation. Some modern systems may make use of highly-sensitive, low-noise digital DC magnetometers in the receiver to directly sample the local magnetic field at its position and orientation. In a traditional coil-based sensor setting, receiver sensitivity may be increased by increasing the frequency of the fields (by Faraday's Law, the EMF is the derivative of the magnetic flux (P) through the sensing coil, which grows linearly with frequency). However, with DC magnetometers, increasing the frequency may not improve the sensitivity of a sensor; a DC magnetometer has a fixed noise level regardless of the sensed frequency of the field.

### SUMMARY

It is an object of the present invention to provide an apparatus, a system, a computer program product, and a method for Electromagnetic Tracking Systems (EMTS) and, more specifically to coil geometries for a transmitter coil, but not exclusively, to transmitter coils.

A coil topographical geometry including two or more armatures and multiple loops wound about the two or more armatures to form two or more coils. One or more fundamental loops of the multiple loops crosses over themselves at one point in the one or more fundamental loops to form a geometrical shape in the space occupied by the coil topographical geometry. The one or more fundamental loops of the multiple loops are configurable to generate one or more fundamental fields [Bfun] that are substantially the same as the fields [Bi(r)] generated by the two or more coils.

The fields [Bi(r)] generated by the two or more coils may be modulated at different frequencies. The fields [Bi(r)] generated by the two or more coils are symmetrical to each other so that the mutual inductance between the two or more coils and one or more other coil is substantially zero. The two or more coils and the one or more other coil may be utilized as a transmitter coil. The transmitter coil located in an electrically conductive loop that surrounds the transmitter coil. The fields [Bi(r)] of the transmitter coil normal to the electrically conductive loop, induces a reduced contribution of distortive electromotive force (EMF) from the electrically conductive loop to the fields [Bi(r)] generated by the transmitter coil.

The transmitter coil responsive to a mutual inductance between the transmitter coil and the loop is substantially zero and may emit the sum of the fields [Bi(r)] with reduced power dissipation in the transmitter coil. The geometrical shape of the two or more coils may be substantially a figure of eight (8) shape. An overall tracking error enables a finding of an optimal design geometry for the transmitter coil.

The coil topographical geometry may further comprise a microcontroller. One or more receivers may be operatively attached to the microcontroller. The one or more receivers in the proximity of the two or more coils, may be configured to sense the fields [Bi(r)] transmitted by the two or more coils. The one or more receivers may be further configured to calculate a position, an orientation and an accurately predicted error estimation of the position and the orientation, in five or six degrees of freedom (DOF) of the one or more receivers. The accurately predicted error estimation may be utilizable in a configuration of a multiple receiver electromagnetic tracking system (EMTS).

A filter may be operatively attached to the one or more receivers, the filter configurable by use of the accurately predicted error estimation to filter noise of the one or more receivers from the measured fields [Bi(r)] transmitted by the two or more coils. Each of the two or more armatures may be a printed circuit board (PCB). The PCB including one or more layer to enable one or more loop to cross over itself at one point in the loop. The loop may be implemented as a PCB trace.

The one or more loops may comprise a first self-filling trace wound clockwise. The first self-filling trace including a first end and a second end. A second self-filling trace adjacent to the first self-filling trace. The second self-filling trace wound counterclockwise. The second self-filling trace including a third end and a fourth end. A trace connects the second end to the fourth end. The first and third ends provide a connection to a signal source.

A method for a coil topographical geometry comprising winding multiple loops around two or more armatures forming two or more coils. One or more points are crossed over in the multiple loops to form one or more fundamental loops in the multiple loops. A geometrical shape is formed responsive to the crossing point in the space occupied by the coil topographical geometry. The one or more fundamental loops of the multiple loops are configured to produce one or more fundamental fields [Bfun]. The one or more fundamental fields [Bfun] are substantially the same as the fields [Bi(r)] produced by the two or more coils. Transmitting from the two or more coils, the fields [Bi(r)] generated by the two or more coils. Sensing with one or more receiver a superposition of fields generated by the transmitting of the fields [Bi(r)]. The sensing is in the space occupied by the coil topographical geometry. Calculating, thereby tracking a position and an orientation of the one or more receiver responsive to the sensing.

The transmitting of the fields [Bi(r)] may be by the two or more coils modulated at different frequencies. The one or more receivers may be configured to hold readings indicative of their positions and orientations in a space relative to the two or more coils. The sensing enables a calculation of a tracking error between the two or more coils and the one or more receivers. The fields [Bi(r)] generated by the two or more coils may be symmetrical to each other, so that the mutual inductance between the two or more coils and one or more other coils are substantially zero.

The two or more coils and the one or more other coils may be utilized as a transmitter coil. The transmitter coil located in an electrically conductive loop that surrounds the transmitter coil. The fields [Bi(r)] of the transmitter coil normal to the electrically conductive loop induces a reduced contribution of distortive electromotive force (EMF) from the electrically conductive loop to the sum of the fields [Bi(r)] generated by the transmitter coil.

Noise of the one or more receivers may be filtered out from the measured fields [Bi(r)] transmitted by the two or more coils. The sensing and the calculating may be responsive to the filtering. The filtering further derives and uses an accurately predicted error estimation of the position and an orientation of the one or more receivers in five or six degrees of freedom (DOF). The accurately predicted error estimation may be utilizable in a configuration of a multiple receiver electromagnetic tracking system (EMTS).

The transmitter coil responsive to a mutual inductance between the transmitter coil and the electrically conductive loop is substantially zero and emits the fields [Bi(r)] with reduced power dissipation in the transmitter coil. The transmitter coil may emit the field at different specific frequencies. The geometrical shape of the two or more coils may be substantially a figure of eight (8) shape. An overall tracking error may enable a finding of an optimal design geometry for the transmitter coil.

According to a first aspect, a configuration to find an optimal geometry for coils for any configuration of electromagnetic tracking and any constraints.

According to a second aspect, an electromagnetic tracking error is defined, discussed and an efficient optimization method for the configuration is described, to minimize the overall electromagnetic tracking error under various constraints. The efficient optimization method is suitable for example to help find the optimal electromagnetic transmitting coils in a planar PCB transmitter setting. The planar PCB transmitter setting may be for a single axis receiver providing five degrees of freedom (5-DOF) tracking, as well as finding the optimal electromagnetic transmitting coils in a 3-coil planar PCB transmitter for a 3-axis receiver for low-noise, power-efficient 6-DOF tracking.

According to a third aspect, a 3-coil planar PCB transmitter geometry that uses the same coil geometry for each of the three coils can be improved upon by use of a self-crossing 3-coil geometry. The overall improvement may be more than 30% in terms of the overall tracking error under power dissipation constraints. The overall improvement also reduces electromagnetic distortion and mutual inductance between the coils by use of the self-crossing 3-coil geometry.

According to a fourth aspect, a transmitter assembly that includes multiple self-crossing coils, differently oriented and/or differently designed to help distinguish the respective magnetic fields that each produces and where the receiver measures the respective magnetic fields.

According to a fifth aspect, coils are modelled by treating magnetic fields generated or transmitted by multiple elements (individual coil loops and/or segments thereof) as if generated by a single fundamental clement (e.g., a coil loop and/or segment). The single fundamental element being modeled as if it is configured and operated to generate a field approximating the total field of the plurality of elements for which single fundamental element substitutes.

The foregoing and other objects are achieved by the features of the independent claims. Further implementation forms are apparent from the dependent claims, the description and the figures.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

In the drawings:

FIG. 1 shows a block diagram of electromagnetic tracking system (EMTS), in accordance with some embodiments;

FIG. 2 shows further details of a crossover coil, in accordance with some embodiments;

FIG. 3 shows details of a non-crossover coil, in accordance with some embodiments;

FIG. 4 shows a diagram of the Biot-Savart formula applied to a single line segment, in accordance with some embodiments;

FIG. 5 shows a PCB coil implementation of a non-crossover coil shown in FIG. 3, in accordance with some embodiments, and its approximation using 3 fundamental loops;

FIG. 6 shows a geometrical comparison between two types of coil configuration, in accordance with some embodiments

FIG. 7 shows printed circuit board (PCB) drawings of a Type-A and self-crossing coil configuration layouts, in accordance with some embodiments;

FIG. 8 shows a PCB coil implementation of a solved coil configuration, in accordance with some embodiments;

FIG. 9 shows another example of a self-crossing coil, in accordance with some embodiments; and

FIG. 10 shows a flow chart of a method, in accordance with some embodiments.

### DETAILED DESCRIPTION

The present disclosure, in some embodiments thereof, relates to Electromagnetic Tracking Systems (EMTS) and, more specifically to coil geometries for a transmitter coil, but not exclusively, to transmitter coils.

By way of introduction aspects of the disclosure below, includes descriptions to find an optimal geometry for coils for any configuration of electromagnetic tracking and any constraints. A notion of electromagnetic tracking error is defined and discussed herein. In addition, an efficient optimization method is described to minimize the overall electromagnetic tracking error under various constraints. The efficient optimization method is suitable for example to help find the optimal electromagnetic transmitting coils in a planar PCB transmitter setting. The planar PCB transmitter setting may be for a single axis receiver providing five degrees of freedom (5-DOF) tracking, as well as finding the optimal electromagnetic transmitting coils in a 3-coil planar PCB transmitter for a 3-axis receiver for low-noise, power-efficient 6-DOF tracking, as shall be demonstrated below. It will be shown that a 3-coil planar PCB transmitter geometry using the same coil geometry for each of the three coils can be improved upon by use of a self-crossing 3-coil geometry. For example, the overall improvement may be more than 30% in terms of the overall tracking error under power dissipation constraints. The overall improvement also reduces electromagnetic distortion and mutual inductance between the coils by use of the self-crossing 3-coil geometry by a method or methods described herein.

Some aspects of the present disclosure may relate to design methods, design features, and/or use methods for transmitter assemblies comprising multiple planar-packaged transmission coils operable to produce mutually distinguishable electrical fields suitable for position tracking of a receiver. In some aspects, the fields are measurable, using the receiver, to provide information indicative of five or more degrees of freedom (DOF) of the position of the receiver. The transmission coils, in some aspects, are optimized for the reduction of one or more of tracking error, power dissipation, electromagnetic distortion, and mutual inductance. In some aspects, a transmitter assembly includes at least one self-crossing coil twisted over itself, and/or wound in self-opposing directions. In some aspects, a transmitter assembly includes multiple self-crossing coils, differently oriented and/or differently designed to help distinguish the respective magnetic fields that each produces where the receiver measures them. In some aspects, coils are modelled by treating magnetic fields generated or transmitted by multiple elements (individual coil loops and/or segments thereof) as if generated by a single element (e.g., a coil loop and/or segment). The single element being modeled as if it is configured and operated to generate a field approximating the total field of the plurality of elements for which it substitutes.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

The present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network, for example, the Internet, a local area network, a wide area network and/or a wireless network.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

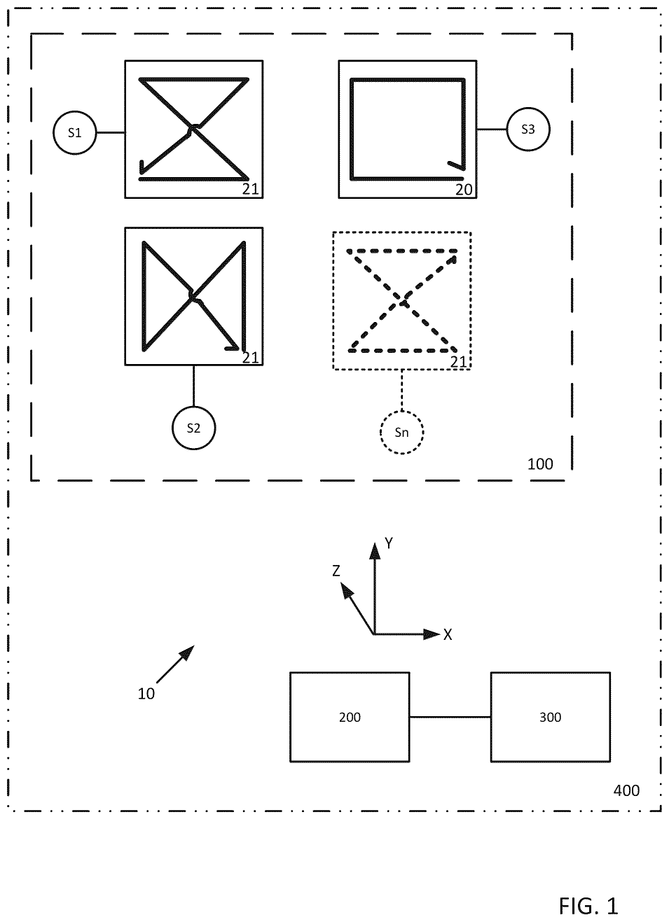

Reference is now made to FIG. 1, which shows a block diagram of a top view of an electromagnetic tracking system (EMTS) 10, in accordance with some embodiments. The top view is shown as a right-hand coordinate system in the XY plane with the Z-axis coming out from the top view towards an observer. EMTS 10 includes a transmitter coil unit 100 that further includes two or more crossover coils 21 and a non-crossover coil 20. Signal sources S1, S2 and Sn connect respectively to crossover coils 21 and signal source S3 to non-crossover coil 20. In proximity to a transmitter coil unit 100 is a receiver 200 configured to measure the magnetic field (B) emitted by transmitter coil unit 100 in three dimensional (3D) space XYZ. The configuration and control of receiver 200 may be by connection of receiver 200 to microcontroller 300. Microcontroller 300 may be implemented as a microprocessor and memory or as a digital signal processor (DSP).

By way of a non-limiting example, a planar printed circuit board (PCB) version of transmitter coil unit 100 may be centered on a table included in an enclosure or treatment room 400. The planar PCB version of transmitter coil unit 100 is discussed in further descriptions that follow. Treatment room 400 may also include an underlying metal frame, or in a medical setting, the underlying metal frame may be placed on a bed with a metal frame surrounding the bed. By way of the non-limiting example, the fields generated by transmitter coil unit 100 may create an electromotive force (EMF) in the metal frames or plates surrounding transmitter coil unit 100. The EMF may create eddy currents flowing in the electromagnetic loop that surrounds transmitter coil unit 100 that generate distorting fields in the same spectrum transmitted by transmitter coil unit 100. Receiver 200 therefore, senses a superposition of the intentionally transmitted magnetic fields of transmitter coil unit 100 plus the distorting fields. The sensing of superposition may not easily allow the intentionally transmitted magnetic fields of transmitter coil unit 100 to be separated from the distorting fields. According to the features of coil geometries for transmitter coil unit 100 discussed in detail below, one of the features are aimed at reducing the magnetic flux (Φ) induced in the metal frames or plates that surround transmitter coil unit 100. Reduced magnetic flux (Φ) induced in the metal frames or plates that surround transmitter coil unit 100 may enable an easier separation or differentiation between the intentionally transmitted magnetic fields of transmitter coil unit 100 and the distorting fields.

In the description above, transmitter coil unit 100 may be described as a set of multiple electromagnetic coils. In descriptions that follow, crossover coils 21 and a non-crossover coil 20 may be represented mathematically and implemented as current loops or current carrying traces on a printed circuit board (PCB) For simplicity, each trace can be mathematically modeled as a single line segment defined by two three-dimensional (3D) vertices having a specific electrical resistance. The electrical resistance of a trace or a coil can be calculated by using the cross-sectional area of a trace, the length of the trace and its material resistivity coefficient. For example, the resistance of a PCB trace can be calculated according to the following formula:

$$R = ρ \frac{L}{w \cdot d}$$

Where ρ is the trace material resistivity coefficient (For example, ρ=1.72×10⁻² Ω·mm in the case of annealed copper at 20° C.), L is the trace length in millimeters, w is the trace width in millimeters and d is the trace thickness in millimeters (which can be derived from the PCB layer copper weight). Resistance R is given in Ohms. It is worth noting that resistivity ρ usually increases with temperature so that resistance R increases with temperature. The increase in resistivity (ρ) is not negligible in the case of high electrical current circuits, where the high current causes power dissipation through the traces resistance, which in turn causes the traces to heat and increase in resistivity until an equilibrium is reached. The increase in resistivity (ρ) is also the case with electromagnetic transmitting coils which may carry significant electrical current to linearly increase the strength of the transmitted electromagnetic field, especially in the setting of low-frequency fields. The phenomena of increased resistivity in transmitting coils should therefore also be accounted when designing crossover coils 21 and a non-crossover coil 20 included in transmitter coil unit 100.

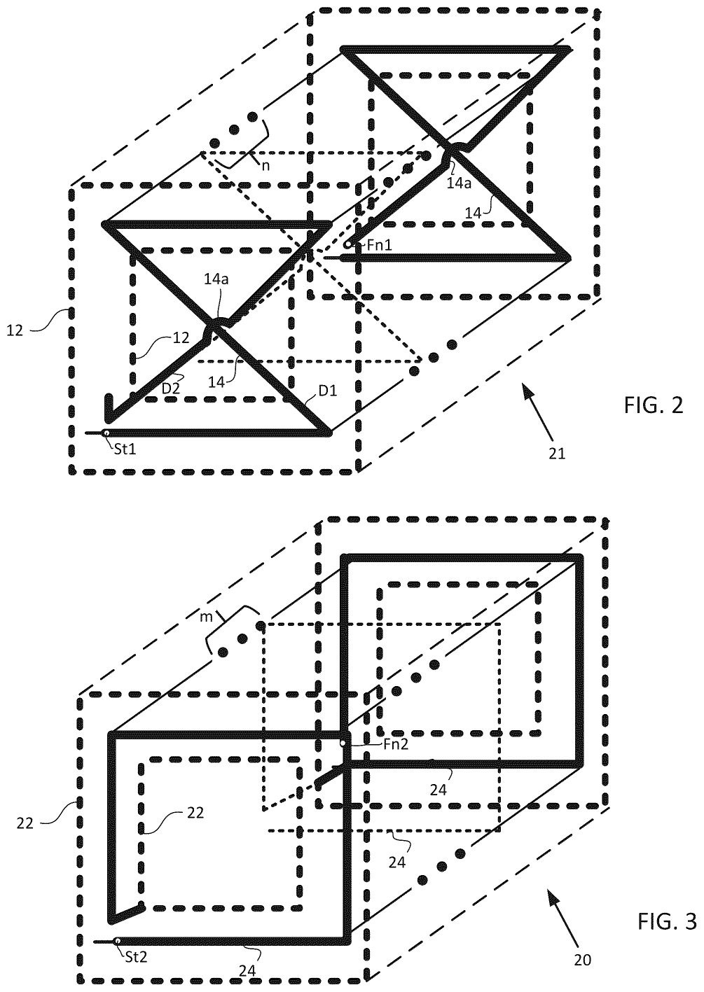

Reference is now made to FIG. 2, which shows further details of a crossover coil 21, in accordance with some embodiments. Crossover coil 21 includes multiple n-turns of a loop 14 starting at starting point St1 and finishing at finish point Fn1. Signal sources S1, S2 (not shown) connect electrically across starting point St1 and finishing at finish point Fn1. Loop 14 starting at starting point St1, is formed on rectangular armature 12. Loop 14 then proceeds to go horizontally right a horizontal distance determined by armature 12 and then crosses back left in an upward direction at approximately forty-five degrees to be just above starting point St1 to form a diagonal D1. Loop 14 then continues again horizontally right according to the horizontal distance. Loop 14 then crosses back left in a downward direction at approximately forty-five degrees to be just above starting point St1 to form a second diagonal D2. In loop 14 going in the downward direction towards starting point St1, loop 14 crosses over itself or more specifically crosses over diagonal D1 to give cross over 14a. Loop 14 may be implemented with an enamel copper wire conductor so that a short circuit situation does not occur at multiple cross overs 14a and between turns of loop 14. Armature 12 supports mechanically multiple loops 14 and may include a ferrite material in order to utilize high magnetic permeability coupled with low electrical conductivity provided by the ferrite material that may help to amplify the transmitted fields of loops 14.

Reference is now made to FIG. 3, which shows details of non-crossover coil 20, in accordance with some embodiments. Non-crossover coil 20 includes multiple m-turns of a loop 24 starting at starting point St2 and finishing at finish point Fn2. Signal source S3 (not shown) connects electrically across starting point St2 and finishing at finish point Fn2. Loop 24 starting at starting point St2, is formed on rectangular armature 22. Loop 24 then proceeds to go horizontally right a horizontal distance determined by the armature 22 and then upwards a vertical distance determined by the armature 22. Loop 24 then continues again horizontally left according to the horizontal distance. Loop 24 then goes vertically downward direction to be just above starting point St2. Loop 24 may be implemented with an enamel copper wire conductor so that a short circuit situation between turns of loop 24. Armature 22 supports mechanically multiple loops 24 and may include a ferrite material in order to utilize high magnetic permeability coupled with low electrical conductivity provided by the ferrite material that may help to amplify the transmitted fields of loops 24. Armatures 12 and 22 may not necessarily be rectangular but may be any polynomial shape so that loop 14 for example may be formed to have a figure of eight (8) shape where the upper and lower horizontal portions of loop 14 are more rounded. Armature 22 may be circular to form circular helical loops as opposed to rectangular helical loops as shown.

In discussions below printed circuit board (PCB) implementations of crossover coils 21 and non-crossover coil 20 may be made where armatures 12 and 22 are implemented using layers of a printed circuit board. Further, loops 14 and 24 may be made from traces etched out from the printed circuit board and interconnection between traces/loops may be by use of multiple layers and vias. Mathematical analysis shown above and below for design of solved coil configuration, and tracking of transmitter coils by a receiver in the description, apply equally to crossover coils 21, non-crossover coil 20 and their PCB implementations described in further detail below.

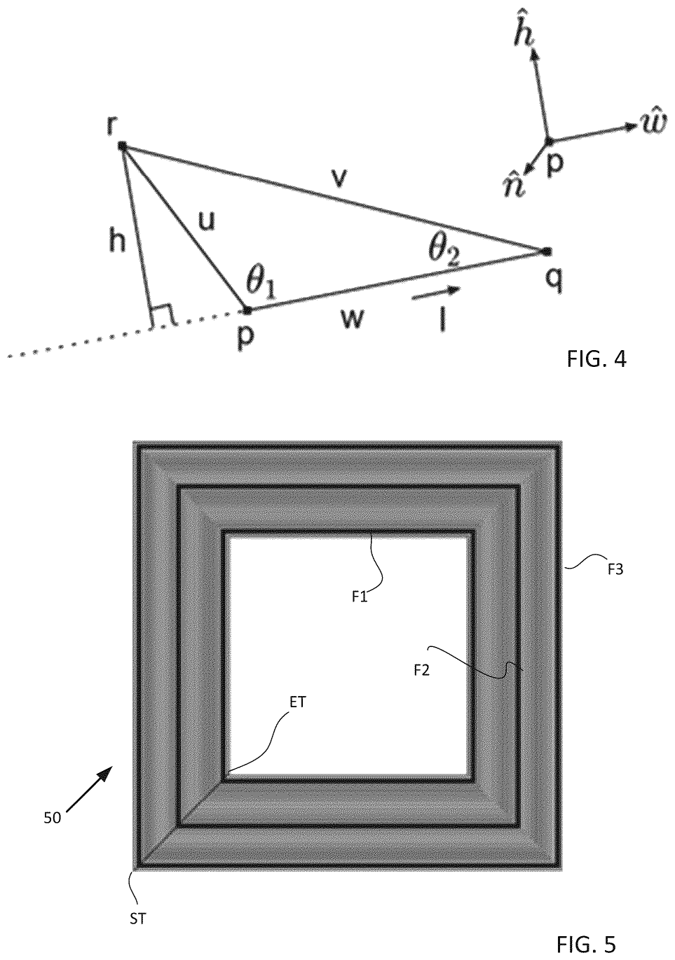

Reference is now made to FIG. 4, which shows a diagram of the Biot-Savart formula applied to a single line segment, in accordance with some embodiments. For a given transmitting coil or trace geometry, the generated magnetic near field can be modeled precisely by using the Biot-Savart equation. The Biot-Savart equation describes the magnetic field (B) generated by a constant electric current. While the current (I) does alternate in an electromagnetic transmitting coil usually in a sinusoidal manner. Because the frequencies (f) of signal sources S1, S2 and Sn are in the kilohertz range. The wavelengths (λ) of signal sources S1, S2 and Sn are in kilometers range that are much larger compared to the PCB size of usually less than 1 meter×1 meter and the desired sensing range. Therefore, the effect on the magnetic near field at the proximity of transmitter coil unit 100 is negligible and the fields can be thought of as static for the sake of applying the Biot-Savart law. By integrating the differential magnetic field elements in the Biot-Savart formula along a line segment, the following analytic formula can be obtained. The analytic formula describes the magnetic field generated by a single constant current carrying line segment [p, q]:

$$B ( r ) = \frac{{μ}_{0}}{4 π} \frac{I}{h} ( cos ( {θ}_{1} ) - cos ( {θ}_{2} ) ) \cdot \hat{n}$$

Where r is the three-dimensional (3D) position in space at which the magnetic field should be computed. μ₀ can be simply taken as the magnetic permeability of vacuum (4π×10⁻⁷ [H/mm]). I is the constant electrical current through the line segment given in Amperes. Distance h is the distance in meters between r and the infinite line (shown by dashed line) passing through the line segment [p, q]. Angle θ₁ is the angle formed between u and w. Angle θ₂ is the angle formed between and w. Vector \hat{n} is the direction of the magnetic field B(r). The direction of the magnetic field B(r) is the normal of triangle [p, q, r] given in Tesla units.

For simplicity, the magnetic permeability uo can be substituted into the formula and the formula can be simplified as follows:

$$B ( r ) = 1 {0}^{2} \frac{I}{h} ( cos ( {θ}_{1} ) - cos ( {θ}_{2} ) )$$

Where h is now taken in millimeters and B is given in [uT] (microtesla units).

Computing the magnetic field generated by a coil that includes multiple line segments is straightforward: each line segment is treated independently and contributes its own magnetic field to the total superposition of all magnetic fields:

$$B ( r ) = {\sum }_{i} {B}_{i} ( r ) = 1 {0}^{2} I {\sum }_{i} \frac{cos ( {θ}_{1}^{i} ) - cos ( {θ}_{2}^{i} )}{{h}_{i}}$$

Where i iterates over all the line segments which belong to the coil. By Kirchhoff's law the current (I) is identical in all the line segments belonging to the same coil, so current (I) is taken outside the sum (Σ). For a coil consisting of many line segments, for example, a PCB coil consisting of 100 rectangular windings, an efficient way may be used to compute the above formula. The efficient way can be achieved for example using vector processing. An example of vector processing is single instruction multiple data (SIMD™) that is a type of parallel processing. Writing the sum (Σ) above using vector operations, provides a significant computational boost over an implementation in a standard computational “for” loop. The significant computational boost may be especially in mathematical software that make use of SIMD™ operations such as MATLAB™, as well as highly optimized compiled C++ code using special mathematical libraries or even using compute unified device architecture (CUDA™) or other parallel computation frameworks. The significant computational boost enables computation of the magnetic field generated by crossover coils 21 and non-crossover coil 20 efficiently and accurately. The significant computational boost may enable overall a finding of an optimal coil geometry for transmitter coil unit 100 and for solving position and orientation in electromagnetic tracking system (EMTS) 10. Each of the operations in the sum above can be easily described using simple vector operations such as vector-by-vector elementwise multiplication or division, elementwise square or square root, etc. For example, the cosine elements can be computed using a dot product between the corresponding vectors divided by the corresponding norms, all done using vector operations.

The electrical current I can be computed for a specific coil geometry depending on its electrical resistance properties given a specific voltage; The total coil resistance R is computed by summing each separate line segment resistance Rᵢ, whereas each Rᵢ is computed as discussed above using each segment's length, width and depth. Once total coil resistance R is computed for the entire coil, electrical current I can be simply computed for a given voltage V using Ohm's law:

$$I = \frac{V}{R} .$$

The total superposition of all magnetic fields B(r), represents the magnetic field in three-dimensional (3D) point r in space in the coordinates of transmitter coil unit 100. However, a receiver such as receiver 200, senses the magnetic field at its position in its local coordinate system. The magnetic field can be sensed and described in the coordinate system of receiver 200, using the following formula:

$${B}_{rx} ( r , Θ ) = {R ( Θ )}^{t} B ( r )$$

Where r is a 3-dimensional vector representing the position of receiver 200 in transmitter coil unit 100 coordinates, Θ=(θₓ, θ_{y}, θ_{z}) is a 3-dimensional vector representing the 3 Euler angles which indicate the orientation of receiver 200 in transmitter coil unit 100 coordinates and R(Θ) is a 3×3 rotation matrix corresponding to Θ which can be obtained using standard formulas. Bᵣₓ(r, Θ) is a 3-dimensional magnetic field in local sensor coordinates which corresponds to a specific transmitting coil. Bᵣₓ(r, Θ) converts between receiver 200 position and orientation (6-DOF) to transmitting coil magnetic field in local receiver 200 coordinates.

In electromagnetic tracking, receiver 200 senses the superposition of fields generated by crossover coils 21 and a non-crossover coil 20. Normally, receiver 200 separates between the fields generated by crossover coils 21 and a non-crossover coil 20 using Discrete Fourier Transform, correlation or other methods, utilizing the fact that crossover coils 21 and a non-crossover coil 20 operate at different very specific designated frequencies. Receiver 200 then holds readings in memory of microcontroller 300 indicative of its position an orientation in space relative to transmitter coil unit 100. Each position and orientation in space should have a different magnetic signature, such that by sensing it, it could be uniquely converted back to position and orientation, usually in real-time.

In a multiple transmitting coils setting that includes crossover coils 21 and a non-crossover coil 20. The magnetic fields of crossover coils 21 and a non-crossover coil 20 are denoted by B(r). The concatenation of all magnetic fields corresponding to crossover coils 21 and a non-crossover coil 20 at position r as a 3×N matrix, where N is the number of transmitting coils and each column of this matrix represents the magnetic field of a single transmitting coil. For example, in a 3-coil transmitter magnetic filed B(r) is a 3×3 matrix where each of its columns corresponds to one of crossover coils 21 or non-crossover coil 20. In an 8-coil transmitter, for example a single-axis receiver 200 magnetic field B(r) is a 3×8 matrix. The magnetic fields at receiver 200 position r and orientation Θ can again be described in a local receiver 200 coordinates using:

$${B}_{rx} ( r , Θ ) = {R ( Θ )}^{t} B ( r )$$

Which is now a full 3×N matrix. While the magnetic fields at receiver 200 position and orientation can be described as a full 3×N matrix, receiver 200 may not be able to sense all 3 components of the field (Bᵣₓ). For example, in the setting of a single-axis receiver 200, receiver 200 only senses the local magnetic field along its Z axis. On the contrary, receiver 200 may only sense the field in the X-Y axes but may also sense along its Z axis. In all these cases, the fields sensed by receiver 200 may be described using the following formula:

$$X ( r , Θ ) = U \cdot {B}_{rx} ( r , Θ )$$

Where U is the calibration matrix of receiver 200. Calibration matrix U is a C×3 matrix, where C (1, 2 or 3) is the number of sensor axes. Calibration matrix U describes how the sensing properties of receiver 200 converts the local magnetic fields at its position and orientation into sensed values. For example, with a perfectly calibrated 3-axes sensor, calibration matrix U=I_{3×3} (the identity matrix) and the sensed values X(r, Θ) are just equal to Bᵣₓ (r, Θ). However, with a single-axis receiver calibration matrix U= (0,0,1) and X(r, Θ) is a 1×N vector (the sensing properties of receiver 200 only senses one projection for each transmitted field along its Z axis). Calibration matrix U can more generally encode the calibration of the sensing properties of receiver 200, for example, with receiver 200 as a single-axis receiver, calibration matrix U=(0,0, gain) where gain describes the sensitivity of the single-axis sensor of receiver 200. Also, in a 3-axis sensor calibration matrix U_{3×3} may describe the nonorthogonality of the 3 different sensing axes which yields the final measurements of the field by receiver 200.

One goal of electromagnetic tracking system (EMTS) 10 is to compute the inverse of X(r, Θ), that is, to convert between magnetic fields sensed in local receiver coordinates with sensor calibration matrix U to a position and orientation (r, Θ) in a 6-DOF setting, or to a position and partial orientation (r, θₓ, θ_{y}) in a 5-DOF setting (in which the roll angle θ_{z} is absent).

Sensed value X(r, Θ) is usually computed by processing a short-time samples window acquired by the magnetic sensor of receiver 200, using methods described above. After sensed value X(r, Θ) is computed with each of its columns corresponding to a separate sensed magnetic field in receiver 200 coordinate system, a solver is used to find the position and orientation (r, Θ). In the most general setting, in order to find the inverse of X(r, Θ) a nonlinear optimization method can be utilized to minimize the following cost function:

$$E ( r , Θ ) = {‖ X ( r , Θ ) - {X}_{0} ‖}^{2}$$

Where X₀ is the sensed magnetic fields matrix (now treated as a flat vector), X(r, Θ) is the predicted magnetic fields matrix (now treated as a flat vector) at position and orientation (r, Θ) computed based on the Biot-Savart model discussed above, and (r, Θ) are the searched parameters for the optimization-the position and orientation of receiver 200. Any type of solver, whether based on polynomials, has a global initialization or refinement iterations or brute force, eventually tries to find position and orientation (r, Θ) which minimize E(r, Θ). Hence, optimizing over E(r, Θ) makes up the optimal (but not necessarily the most computationally effective) solution for electromagnetic tracking.

Cost function E(r, Θ) can be optimized using various nonlinear optimization methods, such as Gradient-Descent, Newton-Raphson, Nelder-Mead, Trust Region, Levenberg-Marquardt, and more. For example, Levenberg-Marquardt makes use of the vector function inside the norm and its Jacobian to compute effective optimization steps, thus reducing the total number of steps in the optimization and improving optimization runtime. Denote:

$$F ( x ) = F ( r , Θ ) = X ( r , Θ ) - {X}_{0}$$

$$J ( x ) = J ( r , Θ ) = {J}_{F} ( r , Θ ) = {( \frac{\partial {F}_{i}}{\partial {x}_{j}} )}_{i , j}$$

Where x=(r, Θ) is the 6-dimensional parameters vector being searched (for 6-DOF tracking), F(r, Θ) is the vector error function and J_{F}(r, Θ) is its Jacobian matrix containing all partial derivative combinations. For N transmitting coils, X(r, Θ) is a 3×N matrix now treated as a 3N×1 vector and J_{F}(r, Θ) is a 3N×6 matrix containing all partial derivative combinations. The Levenberg-Marquardt method (or any other suitable method) uses the Jacobian matrix J_{F}(r, Θ) to compute effective optimization steps of parameters vector x, converging efficiently to a parameters vector x₀=(r₀, Θ₀) which minimizes E(x)=∥F(x)∥² and which represents the solved position and orientation (r₀, Θ₀).

As with B(r) and X(r, Θ), an efficient way must be used to compute J_{F}(r, Θ). This also can be achieved for example using vector processing. Similarly to B(r) (here a 3-dimensional vector representing the magnetic field generated by a single coil), an analytic expression first needs to be derived for J_{B}(r) where

$${J}_{B} ( r ) = {( \frac{\partial {B}_{i}}{\partial {r}_{j}} )}_{i , j}$$

is a 3×3 Jacobian matrix corresponding to a single transmitting coil at point r. For a single line segment, J_{B}(r) can be easily computed in a special whn coordinate system as depicted in FIG. 3. Where ŵ is the direction vector of the line segment [p, q], ĥ is the unit perpendicular between point r and the infinite line through [p, q] and \hat{n}=ŵ×ĥ is the normal of the triangle [p, q, r] (pointing the direction of the magnetic field B(r)), and whereas the coordinate system origin is located at p. r can then be expressed in whn coordinates as r=(r_{w}, rₕ, rₙ) and it follows that:

$$\frac{\partial B}{\partial {r}_{w}} ( r ) = I ( \frac{sin ( {θ}_{1} )}{{‖ u ‖}^{2}} - \frac{sin ( {θ}_{2} )}{{‖ v ‖}^{2}} ) \hat{n}$$

$$\frac{\partial B}{\partial {r}_{h}} ( r ) = ( - \frac{‖ B ( r ) ‖}{h} - I \frac{cos ( {θ}_{1} )}{{‖ u ‖}^{2}} + I \frac{cos ( {θ}_{2} )}{{‖ v ‖}^{2}} ) \hat{n}$$

$$\frac{\partial B}{\partial {r}_{n}} ( r ) = - \frac{‖ B ( r ) ‖}{h} \hat{h}$$

And finally:

$${J}_{B}^{whn} ( r ) = {( \frac{\partial B}{\partial {r}_{w}} ( r ) , \frac{\partial B}{\partial {r}_{h}} ( r ) , \frac{\partial B}{\partial {r}_{n}} ( r ) )}_{3 \times 3}$$

(in whn coordinates)

J_{B}^{whn}(r) can then be easily converted to transmitter coordinates by applying the chain rule with a simple rotation:

$${J}_{B} ( r ) = {J}_{B}^{whn} ( r ) \cdot ( \begin{matrix}- \hat{w} - \\ - \hat{h} - \\ - \hat{n} -\end{matrix} )$$

As with B(r), J_{B}(r) of a coil consisting of multiple line segments is just the sum of all individual J_{B}_{ᵢ}(r) of each line segment. Also, J_{B} can be computed with vector operations, for example, sin(θ₁) and sin(θ₂) can be computed using cross product vector operations divided by vector norms. In addition, B(r) and J_{B}(r) share some computations which enables to compute them both simultaneously with minimal computational effort.

Recall that in a multiple transmitting coils setting, B(r) is the concatenation of all magnetic fields corresponding to all transmitting coils at position r as a 3×N matrix. B(r) can also be represented as a 3N×1 vector, in which case its Jacobian matrix J_{B}(r) is a 3N×3 matrix containing all partial derivative combinations. In the case of X(r, Θ) which for 6-DOF tracking also includes 3 Euler angles indicative of receiver 200 orientation, J_{X}(r, Θ) is represented by a 3N×6 matrix, which is computed as follows:

$$\frac{\partial X ( r , Θ )}{\partial {r}_{i}} = U \cdot {R ( Θ )}^{t} \frac{\partial B ( r )}{\partial {r}_{i}}$$

$$\frac{\partial X ( r , Θ )}{\partial {θ}_{i}} = U \cdot \frac{\partial {R ( Θ )}^{t}}{\partial {θ}_{i}} B ( r )$$

Where rᵢ indicates the x, y, z coordinates of r for i=1,2,3 respectively,

$$\frac{\partial B ( r )}{\partial {r}_{i}}$$

is computed as discussed above,

$$\frac{\partial {R ( Θ )}^{t}}{\partial {θ}_{i}}$$

are the transposed partial derivatives of the rotation matrix relative to the 3 Euler angles and are computed using standard analytic formulas and U is the 3×3 sensor calibration matrix as discussed above. The partial derivatives of X(r, Θ) are concatenated into a final 3N×6 matrix J_{X}(r, Θ).

To conclude, F(r, Θ) is the vector error function to be minimized. It is a 3N×1 vector with the expression: F(r, Θ)=X(r, Θ)−X₀, and its Jacobian J_{F}(r, Θ) is a 3N×6 matrix represented as:

$${J}_{F} ( r , Θ ) = {J}_{X} ( r , Θ )$$

Since X₀ is constant. Both F and its Jacobian J_{F} can be computed very efficiently using vector operations as discussed above. This allows for F(r, Θ) to be optimized very efficiently even for very complex coils which contain many line segments, due to the vectorized computation and the computation sharing between F and J_{F}. The optimization problem can be adjusted to the case of 5-DOF tracking or any kind of receiver 200 structure and calibration by adjusting the sensor calibration matrix U accordingly.

Reference is now made to FIG. 5, which shows a PCB coil 50 implementation of non-crossover coil 20 shown in the previous figures, in accordance with some embodiments. PCB coil 50 consists of fifty rectangular windings that amounts to a total of 200 line segments. In gray are the original traces and in bold are three fundamental loops F1, F2 and F3. The start of the coil shown as the start of trace ST. Trace ST winds inwards to form the rectangular windings of the coil. The end of the coil is shown at the end of the trace ET. The end of the trace ET includes another trace which enable the start of the trace ST and the end of the trace to be connected to signal source S3 (not shown).

Using sensed value X(r, Θ) and its Jacobian matrix at (r, Θ): J_{X}(r, Θ) as described above with respect to FIG. 4. The predicted sensed magnetic fields and their derivatives can be computed efficiently using vector operations to speed up the process of finding the position and orientation (r, Θ) for given magnetic measurements X₀. Finding the position and orientation (r, Θ) for given magnetic measurements X₀ allows using transmitter coil unit 100 with very complex coils geometry. For example, a PCB coil 50 with one hundred rectangular windings per each coil to accurately predict the transmitted fields and being able to invert the transmitted fields into a position and orientation in real-time. However, real-time electromagnetic tracking can run at very high rates (30 Hz, 60 Hz or even 200 Hz and above) and computing X and J_{X} at these high rates for very evolved coil geometries, especially with low-end processors or microprocessors, may be an uneasy task. It is therefore desirable to even further reduce computation times with minimal error.

One possible solution is to use a precomputed grid which describes the generated fields in space. The fields can then be computed in real-time using linear, cubic or any other suitable interpolation methods inside the precomputed grid. However, for increased accuracy the precomputed grid needs to be fairly dense, for example, with steps of no greater than 1 mm. In order to cover a total sensing cube of about 500 mm width, height and depth, 125 mega points need to be saved, with each point representing 3-component field values per each transmitting coil. This accumulates to a huge amount of data that may not fit inside a low-end processor's or microprocessor's memory. In addition, it is sometimes desirable to be able to track even outside the predefined sensing cube, but the precomputed grid method may not offer any easy extrapolation method.

In the Biot-Savart method described above B(r) is written as the sum of 200 individual magnetic fields Bᵢ(r) corresponding to the individual line segments. In order to speed up the computation, the original loop (in gray) is broken into three fundamental loops F1, F2 and F3. The original loop can also be broken to just 1 fundamental loop, or 2, or 4, or greater). With respect to the three fundamental loops F1, F2 and F3 the number of elements in the sum is greatly reduced from 200 elements to just 12, by iterating only over segments belonging to fundamental loops F1, F2 and F3. In order to perform this simplification, a set of fundamental loops F1, F2 and F3 need to be searched to approximate the original fields with minimum error. As it turns out, any densely wound coils can be replaced with just a few such fundamental loops F1, F2 and F3.

In order to find the fundamental loops F1, F2 and F3 for a specific coil, the number of fundamental loops F1, F2 and F3 to be used is first chosen (for example, 3 as in FIG. 5), and each loop is again described using a set of fundamental line segments drawn between vertices. The field generated by the fundamental loops is written as:

$${B}^{fun} ( r , {I}_{i} , {p}_{ij} ) = {\sum }_{i = 1}^{D} {I}_{i} {\sum }_{j = 1}^{4} {B}_{ij} ( r )$$

Where pᵢⱼ is the 3-dimensional j-th vertex of fundamental coil i, Iᵢ is the virtual current through coil i, D is the chosen number of approximating fundamental coils, also thought of as the degree of approximation, Bᵢⱼ(r) is the magnetic field due to the individual j-th fundamental line segment in the i-th fundamental coil (defined by pᵢⱼ and p_{i,mod(j+1,4)}). Note that the number 4 is chosen to describe quadrilateral coils, but can be modified to described different shapes with different levels of geometrical approximation (for example, 3 fundamental line segments to constitute fundamental triangular loops). In order to approximate the original magnetic fields, the positions pᵢⱼ and the virtual currents Iᵢ are optimized as to minimize the following error function:

$$E ( {p}_{ij} , {I}_{i} ) = {\sum }_{k} {E}_{k} = {\sum }_{k} {‖ {B}^{fun} ( {r}_{k} , {I}_{i} , {p}_{ij} ) - B ( {r}_{k} ) ‖}^{2}$$

Where rₖ are multiple points chosen for example on a regular grid inside the sensing range, to ensure good approximation inside the sensing range. Each individual error element Eₖ can be further weighted for example to focus the optimization on the center of the sensing range where accuracy is of greater issue. E(pᵢⱼ, Iᵢ) can be optimized using various nonlinear optimization methods, such as Gradient-Descent, Newton-Raphson, Nelder-Mead, Trust Region, Levenberg-Marquardt, and more. The result of the optimization is a set of fundamental vertex positions pᵢⱼ and virtual currents Iᵢ which provide an optimal approximation of the original coils with just a few fundamental line segments.

An optimal approximation is completely theoretic in that it does not use any measurements, it is the approximation of magnetic fields generated by coils containing many windings by fundamental loops F1, F2 and F3. Approximating coils of the form as in FIG. 5 proves to be very efficient with just a few fundamental loops. With just D=3 (3 fundamental loops F1, F2 and F3) a maximum approximation error of about 0.1% can be achieved in a cubic grid with size slightly greater than that of the original coil, which perfectly covers the sensing range. The approximation error reduces as the distance from the coil increases, which makes it perfect for out-of-grid sensing. Choosing D=3 or slightly greater can speed up computation by a factor of 10-20 times or even more compared to computing the original fields directly, using the original traces, depending on the number of original windings, with insignificant error. The model is saved with just a few parameters (pᵢⱼ, Iᵢ) which does not require huge amounts of memory. Microcontroller 300 or a microprocessor can use B^{fun} for solving position and orientation (r, Θ) using methods described above or any other suitable methods, by just replacing B(r) with B_{fun}(r) in any of the formulas (that is, regarding the magnetic fields as being generated by the fundamental loops with their corresponding virtual currents Iᵢ instead of using the original coils).

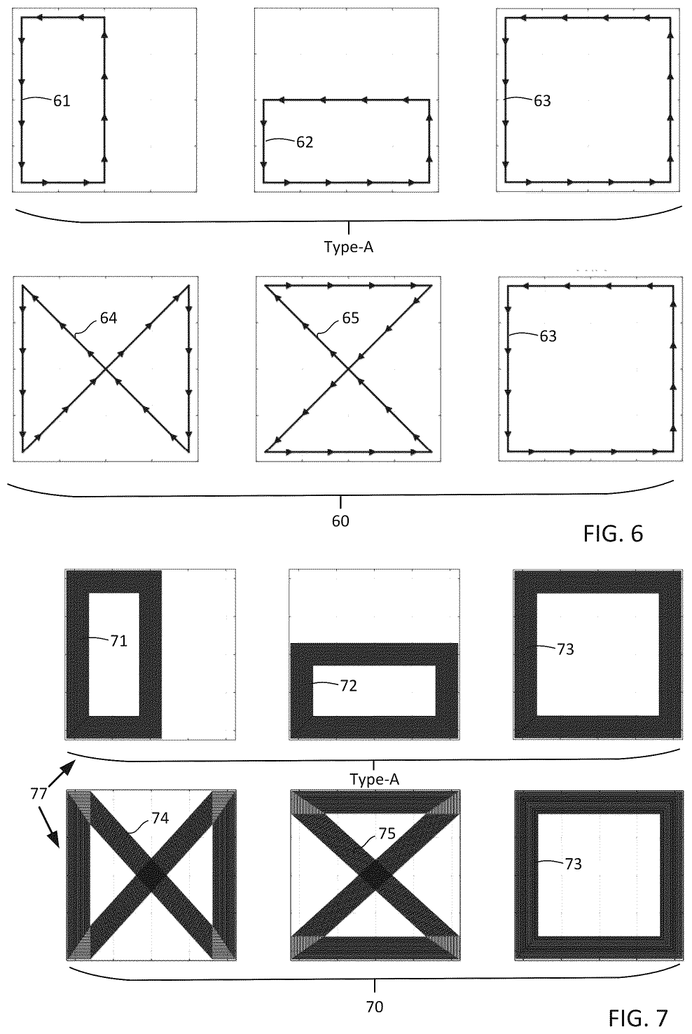

Reference is now made to FIG. 6, which shows a geometrical comparison between two types of coil configuration, in accordance with some embodiments. Coils 61-65 are shown in bold as fundamental loops, which in the descriptions above, enable in general, a configuration of a loop or multiple of loops to provide mathematically a fundamental field approximation B_{fun}(r) from the fundamental loops. Where the fundamental fields B_{fun}(r) from fundamental loops of transmitter coil Type-A, transmitter coil 100 or self-crossing coil configuration 60 mathematically speaking, are substantially the same as the fields Bᵢ(r) transmitted by transmitter coil Type-A, transmitter coil 100 or self-crossing coil configuration 60 respectively. Both fields may be modulated (frequency modulation for example) at different frequencies and transmitted by transmitter coil Type-A, transmitter coil 100 or self-crossing coil configuration 60 with respect to a common point (not shown) in three dimensional (3D) space XYZ. In other words, the field B_{fun}(r) may be modulated at different frequencies, to enable a configuration step of self-crossing coil configuration 60 prior to the modulations transmitted as modulated fields Bᵢ(r) from self-crossing coil configuration 60 subsequent to the configuration step. The fields from self-crossing coil configuration 60 can be separated in receiver 200, for example using a discrete furrier transform (DFT).

Using the methods described above, an optimal geometry was found for a 3-coil transmitter PCB with quadrilateral loops under power dissipation and PCB dimensions constraints to give a solved configuration self-crossing coil configuration 60. Each coil was modeled using a single fundamental loop (4 vertices) and a combination of global and local optimization was used to find the global minima for the coils geometry. FIG. 6 shows two configurations, the Type-A 3-coil transmitter (1^{st} row) versus self-crossing coil configuration 60 (2^{nd} row). While coil 63 is identical between the two configurations, coils 64 and 65 are “self-crossing” in self-crossing coil configuration 60. In terms of the overall tracking error, self-crossing coil configuration 60 shows an improvement compared to the Type-A configuration, for example, over 30% improvement under power dissipation constraint. The over 30% improvement means that a 3-coils transmitter with the self-crossing coils configuration such as self-crossing coil configuration 60 will yield smaller (for example, 30% smaller) tracking error in the sensing region of interest by receiver 200 compared to a Type-A 3-coils transmitter with the same power dissipation. The smaller tracking error and reduced power dissipation of self-crossing coil configuration 60 in a comparison with transmitter coil Type-A is because the magnetic field generated by a self-crossing coil(s) conveys more additional information on top of coil 63 of self-crossing coil configuration 60. Whereas in the Type-A configuration coils 61 and 62 are not much different from coil 63 in the Type-A configuration.

Reference is now made to FIG. 7, which shows printed circuit board (PCB) drawings 77 of a Type-A and self-crossing coil configuration 70 layouts, in accordance with some embodiments. Coil 73 is a PCB implementation of non-crossover coil 20 shown in the previous figures and coils 74 and 75 implementation of crossover coil 21 shown in the previous figures. PCB drawings 77 are shown of the Type-A and self-crossing configurations with 50 windings. Coils 71 and 72 are formed by two rectangular coils 71 and 72 that converge toward the center of each rectangular shape form with each winding. The square outline layout for each coil may determine the position and space and orientation between coils 71, 72 and 73 of the Type-A and self-crossing configurations such as self-crossing coil configuration 60 and self-crossing coil configuration 70 layout. In order to implement a self-crossing configuration in a PCB for coils 74 and 75 in self-crossing coil configuration 70, two PCB layers can be used for coils 74 and 75 to be able to cross on each winding without intersecting itself and causing a short circuit. Avoiding the short circuit can be achieved for example by placing a single via per each winding to switch between layers before intersecting at the PCB center, or by dividing coils 74 and 75 into two or more separate sub-coils. The same can be applied for, triangular or rectangular coils where each sub-coil is oppositely wound on a separate layer and the two or more sub-coils are connected in series using one or more vias. Due to PCB manufacturing capabilities, the traces may have width and spacing constraints, so the traces may converge toward the PCB center with each winding as shown with coils 71, 72 and 73, which affects the resulting magnetic field of each coil.

In order to maximize the additional “information” contributed by each transmitting coil, different transmitting coils should have different geometry. To quantify this, the overall tracking system error is defined as the mean local tracking error on a set of positions and orientations in space (for example, over a grid inside the sensing range of interest). Formally, the overall tracking error is defined by averaging the local tracking error covariance matrices over a set or grid of points and orientations of interest, which results in a single overall tracking error covariance matrix. For example, overall tracking error may be defined as follows:

$${C}_{tot} = \frac{1}{K} {\sum }_{k = 1}^{K} {C}_{k} = \frac{1}{K} {\sum }_{k = 1}^{K} C ( {r}_{k} , {Θ}_{k} )$$

Here rₖ is a set of K points in space, for example in a regular grid inside the sensing region of interest, and the orientations Θₖ may be taken simply as the identity orientations, as random orientations or as other orientations of interest. Overall tracking error Cₜₒₜ provides an error estimation for the complete system. Overall tracking error Cₜₒₜ depends on receiver 200 sensor noise and on the conversion process between time-series magnetic samples to separate field readings (both affect C_{X}_{₀}). Overall tracking error Cₜₒₜ depends on the electrical transmission current (which directly affects each of the Jacobian matrices). Overall tracking error Cₜₒₜ depends on the coils geometry (which affect the Jacobian matrices) and overall tracking error Cₜₒₜ depends on the chosen points and orientations of interest rₖ, Θₖ. As always, Cₖ can be weighted differently inside the sum to focus the error for example at the center of the sensing range of interest.

Since overall tracking error Cₜₒₜ provides an accurate error estimation for any complete tracking system of arbitrary transmitting coils and receiver 200, it serves as a powerful tool for evaluating any given system design before production. For example, overall tracking error Cₜₒₜ can be given for transmitter coil unit 100 and with an exact PCB drawing geometry, with exact currents and receiver sensor noise estimations, and it would yield the final overall expected tracking error. This allows comparing between different designs while taking constraints such as manufacturing costs, dimensions, power dissipation or other into consideration. Being able to estimate the overall tracking error also enables to find optimal coil geometries under any custom constraints by means of global and/or local optimization methods.

Regardless, computing overall tracking error Cₜₒₜ for each of the actual configurations shows that the self-crossing configurations; self-crossing coil configuration 60 and self-crossing coil configuration 70 layout are still better. For example, about 30% better in terms of reduced tracking error and reduced power dissipation than the Type-A configuration. The self-crossing PCB implementation given in FIG. 7 is just one among many possible different drawings, where each implementation may use a different number of windings, different trace width and spacing among other implementation features. With each specific configuration, Cₜₒₜ can be computed for the specific drawing (with or without using Fundamental Loops approximation), thus provide accurate prediction for the tracking error of the manufactured PCB transmitter. Cₜₒₜ is then used for assigning a cost for each possible PCB transmitter, enabling a designer to choose the best PCB drawing among several possibilities.

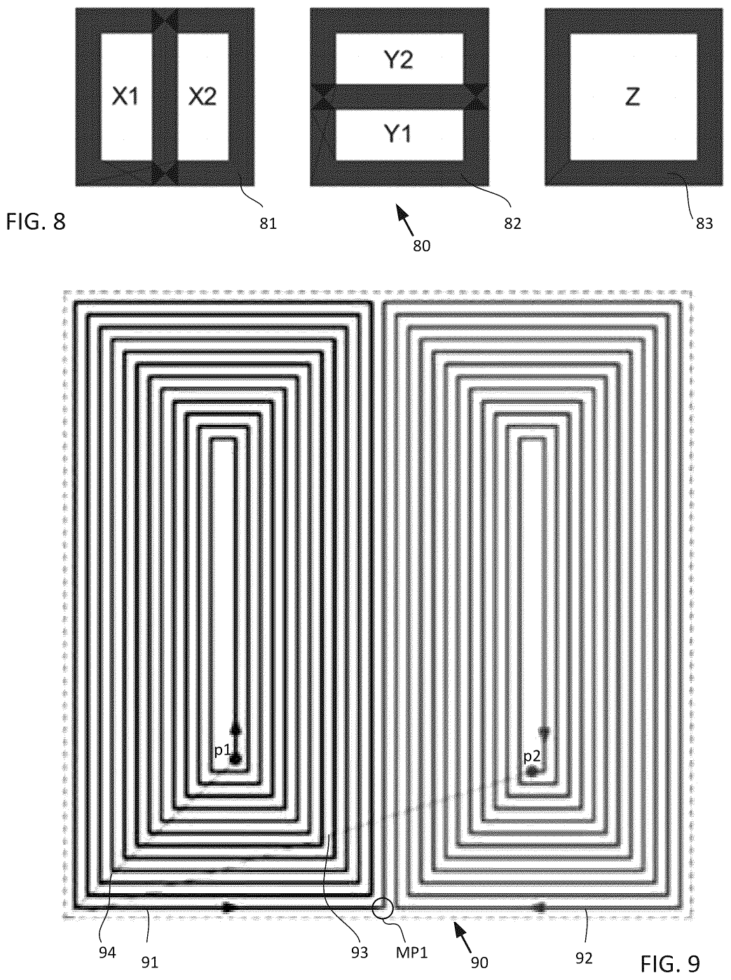

Reference is now made to FIG. 8, which shows a PCB sub-coil implementation of solved coil configuration 80, in accordance with some embodiments. Coil 83 is a PCB implementation of non-crossover coil 20 shown in the previous figures and coils 81 and 82 are implementations of crossover coil 21 shown in the previous figures. The PCB sub-coil implementation of cross over coil 81 is by winding two separate sub-coils X1 and X2 in opposite directions in two separate layers of the PCB and connecting them in series using one or more vias. Similarly for coil 82, by winding two separate sub-coils Y1 and Y2 in opposite directions in two separate layers of the PCB and connecting them in series using one or more vias. Coil 81 is the same as coil 82 but is positioned rotated at ninety degrees relative to coil 82. Coils 81, 82 demonstrate a self-crossing implementation using two sub-coils wound in opposite directions connected in series. Coil 81 consists of two vertical sub-coils X1 and X2 connected in series: Sub-coil X1 wound around the left half of the PCB in a first layer L1, sub-coil X2 wound around the right half of the PCB in a second layer L2. The two sub-coils are connected in series using a single via. Similarly, Coil 82 consists of two horizontal sub-coils Y1 and Y2 connected in series. A self-crossing coil may then be defined more generally as a coil wound in two opposite directions or indirectly by connecting two sub-coils wound in opposite directions in series, as shown.

Reference is now made to FIG. 9, which shows another example of a self-crossing coil 90, in accordance with some embodiments. Self-crossing coil 90 may be drawn on a PCB in a single layer in a Peano curve fashion. The left sub-coil 91 begins at source point p1. The sub-coil 91 is wound in counterclockwise direction until reaching the PCB border. Sub-coil 91 then merges with right sub-coil 92 at merge point MP1. Right sub-coil 92 winds in clockwise direction until reaching terminal point p2. Vias may be placed at source point p1 and terminal point p2 to connect self-crossing coil 90 with other circuitry or to connect its two sub-coils through a second PCB layer. Coil 83 is the same as coils 24, 50 and 73 described above.

Another advantage of the self-crossing configurations described above with respect to cross over coils 14, 74, 75, 81 and 82 is that they are highly symmetric. It is usually desirable that a tracking system such as electromagnetic tracking system (EMTS) 10 will be homogenous inside the sensing region of interest, in the sense that the local tracking errors will spread uniformly and symmetrically through space. With the Type-A configuration this is not the case, since the generated fields do not possess any special symmetry (for example, they do not share the same center). The self-crossing configuration as shown for example in FIGS. 2, 7, & 8, as well as a 3-coil configuration derived from FIG. 9, possesses a high degree of symmetry, with all 3 coils sharing the same center. The symmetry is reflected in the generated fields and can be expressed using the following symmetry equations:

$${B}_{1} ( α x , β y , γ z ) = diag ( γ , αβγ , α ) \cdot {B}_{1} ( x , y , z )$$

$${B}_{2} ( α x , β y , γ z ) = diag ( αβγ , γ , β ) \cdot {B}_{2} ( x , y , z )$$

$${B}_{3} ( α x , β y , γ z ) = diag ( αγ , βγ , 1 ) \cdot {B}_{3} ( x , y , z )$$

$$Where$$

$$α , β , γ = \pm 1.$$

$$Also :$$

$${B}_{1} ( y , x , z ) = ( \begin{matrix}0 & 1 & 0 \\ 1 & 0 & 0 \\ 0 & 0 & 1\end{matrix} ) {B}_{2} ( x , y , z )$$

$${B}_{3} ( y , x , z ) = ( \begin{matrix}0 & 1 & 0 \\ 1 & 0 & 0 \\ 0 & 0 & 1\end{matrix} ) {B}_{3} ( x , y , z )$$

From the symmetry equations, it follows that the mutual inductance between any pair of coils is approximately zero, which makes the self-crossing configurations described above almost mutual inductance free. Self-crossing configuration almost mutual inductance free, simplifies the electromagnetic calibration process of transmitter coil units that include the self-crossing configurations. Further, the electrical driving mechanism to the transmitter coil units is simplified, as crosstalk between different coils becomes negligible.

One other important advantage of the self-crossing configuration relates to electromagnetic distortion. Planar electromagnetic transmitters are usually centered on a table, which might have an underlying metal frame, or in a medical setting may be placed on a patient's bed with a metal frame surrounding it. In these cases, the fields generated by transmitter coil unit 100 might create EMF (Electromotive Force) in the metal frames or plates surrounding transmitter coil unit 100, which may create eddy currents flowing in a loop around transmitter coil unit 100, generating distorting fields in the same spectrum transmitted by transmitter coil unit 100. Receiver 200 then senses a superposition of the intentionally transmitted fields plus the distorting fields which may not be easily separated one from the other.

In order to reduce the distorting field it is necessary to reduce the eddy currents in conductive frames or plates around transmitter coil unit 100. The reduction in the eddy currents may be achieved by reducing the magnetic flux (P) through these conductive loops. Since these conductive loops are usually aligned with transmitter coil unit 100, it is necessary to reduce the 2 component of the magnetic flux (Φ). Where \hat{z} is the normal to the PCB plane of self-crossing coil configurations 70, 80, and 90. By symmetry, it was shown that the mutual inductance between the three coils of self-crossing coil configurations 70, 80, and 90 are zero. Similarly, the mutual inductance between self-crossing coil configurations 70, 80, and 90 and any centered loop, surrounding transmitter coil unit 100 is zero, therefore the magnetic flux (Φ) of Coils 1, 2 through any centered conductive frame surrounding transmitter coil unit 100 is zero. This means that in the self-crossing configuration of self-crossing coil configurations 70, 80, and 90, do not commonly generate any significant distorting field, due to eddy currents. This advantage is highly significant for tracking systems where accuracy is crucial, for example in the medical field. Exploiting this feature can save the need for a special electromagnetic mapping process, for example for electromagnetic tracking system (EMTS) 10 used in the medical field.

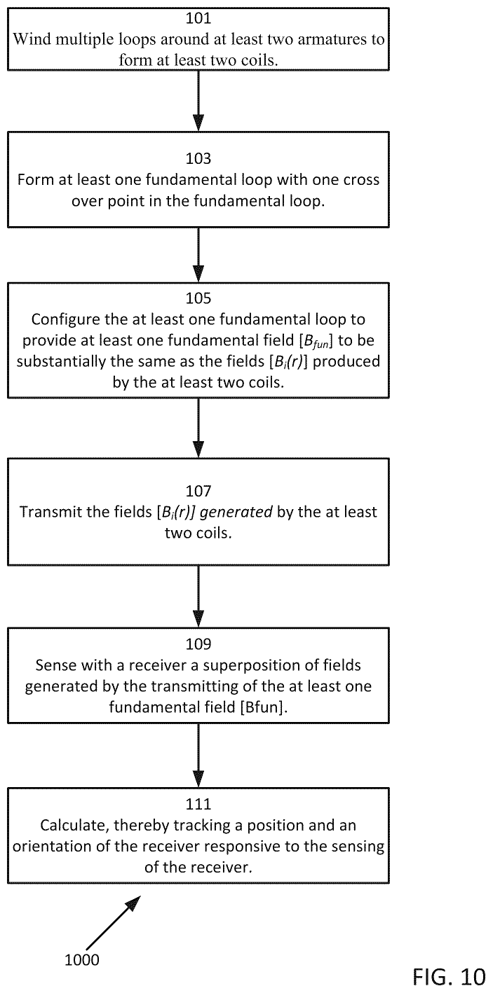

Reference is now made to FIG. 10, which shows a flow chart of a method 1000, in accordance with some embodiments. At step 101, included in a design process of the formation of loops utilized by transmitter coil unit 100 and planar PCB implementations for self-crossing coil configurations 70, 80, and 90 that may be used in transmitter coil unit 100. Transmitter coil unit 100 may include a non-planar implementation using two cross over coils 21 (step 103 and description above) and one non-cross over 20 or planar PCB implementations for self-crossing coil configurations 70, 80, and 90. Implementations of crossover coils 21 (step 103) and non-crossover coil 20 may be made where armatures 12 and 22 are implemented using layers of a printed circuit board. Further, loops 14 and 24 may be made from traces etched out from the printed circuit board and interconnection between traces/loops may be by use of multiple layers and vias. For the purpose of an evaluation between transmitter coil 100 and Type-A in all cases. A number of fundamental loops may form a fundamental electromagnetic field B_{fun}(r) to approximate the fields Bᵢ(r) of each of the coils in solved coil configurations 70, 80, 90 and transmitter coil Type-A at transmit step 107.

At configuration (step 105), in order to find the fundamental loops (as shown in FIG. 5 and FIG. 6) for a specific coil. The number of fundamental loops to be used is first chosen (for example, 3 as in FIG. 5), and each loop is again described mathematically using a set of fundamental line segments drawn between vertices. The fundamental fields B_{fun}(r) from fundamental loops of transmitter coil Type-A, transmitter coil 100 or self-crossing coil configurations 60, 70 and 90 are substantially the same as the fields Bᵢ(r) transmitted by transmitter coil Type-A, transmitter coil 100 or self-crossing coil configurations 60, 70 and 90 respectively. Both fields may be modulated (frequency modulation for example) at different frequencies and transmitted by transmitter coil Type-A, transmitter coil 100 or self-crossing coil configurations 60, 70 and 90 with respect to a common point (not shown) in three dimensional (3D) space XYZ. As described above with respect to transmitter coil Type-A, transmitter coil 100 or self-crossing coil configurations 60, 70 and 90, may be optimized for the reduction of one or more of tracking error, power dissipation, electromagnetic distortion, and mutual inductance that may be included in configuration step 105.

The approximation of fields Bᵢ(r) by fundamental fields B_{fun}(r) may enable reduced and simplified computations for both configuration step 105 and real time sensing and tracking by receiver 200/microcontroller 300 of transmitted fields Bᵢ(r) from self-crossing coil configurations 60, 70 and 90 and tracking of receiver 200. Therefore, microcontroller 300 or a microprocessor can be used for solving position and orientation (r, Θ) of receiver 200 at configuration step 105 and real time sensing and tracking by receiver 200/microcontroller 300 to solve position and orientation (r, Θ) of receiver 200. Therefore, using methods described above or any other suitable methods, at configuration step 105 may be by just replacing Bᵢ(r) with B_{fun}(r) in any of the formulas in regards to magnetic fields as being provided by the fundamental loops with their corresponding virtual currents Iᵢ. Instead of using computations at both configuration step 105 and real time sensing and tracking by receiver 200/microcontroller 300 that use all the loops of a specific coil.

Therefore, at step 105, an optimal geometry may be found for a 3-coil transmitter PCB, under power dissipation and PCB dimensions constraints to give solved configuration self-crossing coil configurations 60, 70 and 90. While coils 63, 73 are identical between configuration type-A and self-crossing coil configurations 60 and 70. Coils 64, 74 and 65, 75 are “self-crossing” in self-crossing coil configurations 60 and 70. In terms of the overall tracking error, self-crossing coil configurations 60 and 70 show an improvement compared to the Type-A configuration, for example, over 30% improvement under power dissipation constraint. The over 30% improvement means that a 3-coils transmitter with the self-crossing coils configuration such as self-crossing coil configurations 60 and 70 will yield smaller (for example, 30% smaller) tracking error in the sensing region of interest by receiver 200 compared to a Type-A 3-coils transmitter with the same power dissipation. The smaller tracking error and reduced power dissipation of self-crossing coil configurations 60 and 70 in a comparison with transmitter coil Type-A is because the magnetic field generated by a self-crossing coil(s) conveys more additional information on top of coils 63 and 73 of self-crossing coil configurations 60 and 70. Whereas, in the Type-A configuration coils 61 and 62 are not much different from coil 63 in the Type-A configuration.

It is usually desirable that a tracking system such as electromagnetic tracking system (EMTS) 10 will be homogenous inside the sensing region of interest, in the sense that the local tracking errors will spread uniformly and symmetrically through space. With the Type-A configuration this is not the case, since the generated fields do not possess any special symmetry for example, they do not share the same center. The self-crossing configuration as shown for example in FIGS. 2, 7, & 8, as well as a 3-coil configuration derived from FIG. 9, possesses a high degree of symmetry, with all 3 coils sharing the same center.

The local tracking error at position and orientation (r, Θ) is defined as the optimal solver's jitter (or noise) in position and orientation for a given sensor measurement noise of a receiver and for a given transmitter current through a coil. As the electrical transmission current increases, the generated magnetic field (step 107) increases in strength and the solver's position and orientation noise decreases proportionally. Equivalently, as the magnetic sensor noise decreases, the solver's position and orientation noise also decreases proportionally.

From the symmetry equations (B₁, B₂ and B₃) shown above for the evaluation between transmitter coil 100 and Type-A. It follows that the mutual inductance between any pair of coils for transmitter coil 100 at step 107 is approximately zero, which makes the self-crossing configurations described above almost mutual inductance free. Self-crossing configuration almost mutual inductance free, simplifies the electromagnetic calibration process of transmitter coil units 100 that include the self-crossing configurations. Further, the electrical driving mechanism to the transmitter coil units 100 are simplified, as crosstalk between different coils becomes negligible.

One other important advantage of the self-crossing configuration relates to electromagnetic distortion. Planar electromagnetic transmitters are usually centered on a table, which might have an underlying metal frame, or in a medical setting may be placed on a patient's bed with a metal frame surrounding it. In these cases, the fields generated by transmitter coil unit 100 might create EMF (Electromotive Force) in the metal frames or plates surrounding transmitter coil unit 100, which may create eddy currents flowing in a loop around transmitter coil unit 100, generating distorting fields in the same spectrum transmitted by transmitter coil unit 100.

Receiver 200 senses (step 109) a superposition of the intentionally transmitted fields plus the distorting fields which may not be easily separated one from the other if a transmitter coil Type-A at transmit step 107 is. In order to reduce the distorting field it may be necessary to reduce the eddy currents in metal frames or plates around transmitter coil unit 100. The reduction in the eddy currents may be achieved by reducing the magnetic flux (Φ) through these metal loops that may also include chassis 400. Since these metal loops are usually aligned with transmitter coil unit 100, it is necessary to reduce the 2 component of the magnetic flux (Φ). Where 2 is the normal to the PCB plane of self-crossing coil configurations 70, 80, and 90. By symmetry described above, it was shown that the mutual inductance between the three coils of self-crossing coil configurations 70, 80, and 90 are zero. Similarly, the mutual inductance between self-crossing coil configurations 70, 80, and 90 and any centered loop such as chassis 400, surrounding transmitter coil unit 100 is zero, therefore the magnetic flux (Φ) of the two cross over coils through any centered metal frame surrounding transmitter coil unit 100 is zero. This means that in the self-crossing configuration of self-crossing coil configurations 70, 80, and 90, do not commonly generate any significant distorting field. This advantage is highly significant for tracking systems where accuracy is crucial, for example in the medical field. Exploiting this feature can save the need for a special electromagnetic mapping process, for example for electromagnetic tracking system (EMTS) 10 used in the medical field.

In electromagnetic tracking (step 111), receiver 200 senses the superposition of fields generated or transmitted at step 107 by transmitter coil unit 100. Normally, receiver 200 separates between the fields generated transmitter coil unit 100 using Discrete Fourier Transform, correlation or other methods, utilizing the fact that transmitter coil unit 100 operate at different very specific designated frequencies. Receiver 200 then holds readings in memory of microcontroller 300 indicative of its position and orientation in space relative to transmitter coil unit 100. Each position and orientation in space should have a different magnetic signature, such that by sensing the different magnetic signature. The different magnetic signature could be uniquely converted back to position and orientation, usually in real-time.

Decreasing overall tracking error Cₜₒₜ can be achieved by increasing the transmission currents in solved PCB coil configurations 70, 80, and 90, but this also leads to increase in total power dissipation of transmitter coil unit 100 that includes solved PCB coil configurations 70, 80, and 90. In a low-power system it is desirable to bring power consumption to minimum while retaining overall tracking error Cₜₒₜ. With the self-crossing configurations of solved PCB coil configurations 70, 80, and 90, power can be reduced (for example, by more than 30%) while retaining the same tracking error Cₜₒₜ, compared to the Type-A configuration. This may be highly desirable for example for a USB powered or battery powered transmitter coil unit 100. Increased power may also cause heating of transmitter coil unit 100 and strong magnetic fields may alert safety concerns of a user or a patient. For all those reasons it is advantageous to reduce transmitter coil unit 100 power while maximizing transmitter coil unit 100 efficiency, as demonstrated by the self-crossing configurations of solved PCB coil configurations 70, 80, and 90, through overall tracking error Cₜₒₜ.

More formally, the local tracking error at position and orientation (r, Θ) shall be defined using C(r, Θ), the covariance matrix of the random variable (r(X), Θ(X)), where X is a random variable describing magnetic field measurements at position and orientation (r, Θ). In the case of 6-DOF tracking, (r, Θ) is 6-dimensional and local tracking error estimation C(r, Θ) is a 6×6 matrix. The magnetic field measurements X is then a 3×3 matrix or a 9×1 vector with some known 9×9 covariance matrix C_{X}_{₀}·C_{X}_{₀}, may encode the realistic sensor noise as well as the noise resulting by the process of converting sequential time series magnetic samples into 3 or more separate magnetic fields for example using DFT or correlation methods. Covariance matrix C_{X}_{₀} can be estimated directly by an offline recording of magnetic samples at an arbitrary position and orientation, applying the Discrete Furrier Transform (DF) or a correlation process and computing the covariance matrix of the processed measurements, or covariance matrix C_{X}_{₀} can be predicted theoretically using standard formulas. In most cases, covariance matrix C_{X}_{₀} is a fairly diagonal matrix representing the sensor noise, and is independent of an exact position and orientation of the sensor in space. The values on its diagonal increase in correlation with the magnetic sensor's noise, and decrease as the DFT or correlation window is made larger.

As described before, X(r, Θ) can be modeled to predict the sensed magnetic fields at a certain position and orientation (r, Θ). Considering position and orientation (r, Θ) as a 6-dimensional random variable (in the case of 6-DOF tracking), the covariance matrix of X(r, Θ) can also be computed using its Jacobian matrix at position and orientation (r, Θ):

$${C}_{X} ( r , Θ ) = {J}_{X} ( r , Θ ) \cdot C ( r , Θ ) \cdot {J}_{X}^{t} ( r , Θ )$$

But as discussed above, C_{X}(r, Θ) is invariant of position and orientation (r, Θ) and is just equal to covariance matrix C_{X}_{₀} at each position and orientation in space. Therefore local tracking error estimation:

$$C ( r , Θ ) = {J}_{X}^{+} ( r , Θ ) \cdot {C}_{{X}_{0}} \cdot {{J}_{X}^{+} ( r , Θ )}^{t}$$

Where J_{X}⁺ is the pseudo-inverse matrix of J_{X} and is defined by: J_{X}⁺=(J_{X}^{t}J_{X})⁻¹J_{X}^{t} (with 6-DOF tracking J_{X} is 9×6 and J_{X}⁺ is 6×9). The formula for the local tracking error C(r, Θ) allows a converting between a known sensor covariance matrix C_{X}_{₀} to a solver's 6×6 covariance matrix at any position and orientation in space. The converting allows to predict the optimal solver's covariance matrix based solely on the known sensor covariance.

For example, when the squared sensor noise decreases, C_{X}_{₀} decreases proportionally and local tracking error estimation C(r, Θ) also decreases proportionally as predicted. On the other hand, when transmitter coil unit 100 current increases, B(r) increases proportionally, then X(r, Θ) also increases in proportion to B(r) and finally J_{X}(r, Θ) increases in proportion with X(r, Θ). Then J_{X}⁺(r, Θ) decreases proportionally and local tracking error estimation C(r, Θ) decreases with a squared proportion, as expected.

The formula for the local tracking error C(r, Θ) can be computed very efficiently in real-time. C_{X}_{₀} reflects the true sensor noise and may be computed and saved in a sensor pre-calibration step (for example, in a factory calibration). The Jacobian matrix J_{X}(r, Θ) is computed repeatedly and used during position and orientation solver iterations, for example in a Levenberg-Marquardt optimization setting, so that its final computed value can be shared and used for C(r, Θ) error computation.

Local tracking error estimation C(r, Θ) can be outputted by electromagnetic tracking system (EMTS) 10 alongside with position and orientation (r, Θ) for outside use of the local tracking error estimation. For example, a user of electromagnetic tracking system (EMTS) 10 may be given a 6-DOF result of (x, y, z)±(∈ₓ, ∈_{y}, ∈_{z}), (α, β, γ)±(∈_{α}, ∈_{β}, ∈_{γ})—a position and orientation and their accurately predicted error estimation which can be used for example in the setting of a multiple sensors tracking system. For example, by placing multiple sensors on a rigid body, the rigid body's position and orientation can be computed or calculated by averaging the positions and orientations of the individually tracked sensors, taking error estimations into account as to lower the averaging weights of poorly tracked sensors. In another use, multiple sensors can be placed on a human's body to track the human skeleton in real-time. The individually tracked sensors may be incorporated in an inverse kinematics scheme where each tracked sensor contributes some constraint on the fully tracked body. Then, for example, in the case of a large position error estimation for a certain skeleton but a better orientation estimation, as reported by electromagnetic tracking system (EMTS) 10. The better orientation estimation may be used in the complete inverse kinematics model at the exact timeframe, or the multiple position and orientation can be weighted differently depending on their relative errors, as reported by electromagnetic tracking system (EMTS) 10. In yet another use of the local error estimation, a set of multiple sensors may be mount inside a catheter, where the catheter's full shape needs to be recovered in real-time. The catheter's full shape can be described using a spline, a polynomial or any other model. Each of the individually tracked sensors contributes a position and orientation constraint or equation to the full catheter's shape. The constraint is weighted with its equivalent error estimation so that noisy sensors affect less on the finally recovered full catheter shape.

Another use of the local tracking error estimation C(r, Θ) may be by constructing an optimal filter for the tracked position and orientation noise based on its exact covariance matrix. For example, under certain conditions, Kalman Filter (linear and extended) provides optimal filtering for noisy measurements, assuming some process model and noise and given the noisy measurements' covariance matrix. It is possible, for example, to construct a Kalman Filter for some tracked receiver 200 with states vector: (r, q, \dot{r}, ω), where r is receiver 200 position, q is receiver 200 orientation (expressed as a quaternion), \dot{r} is receiver 200 velocity and ω is receiver 200 angular-velocity. Deviation from constant velocities, e.g. existence of linear and angular accelerations, can be modeled, for example, as Kalman process noise, with corresponding process covariance matrices. By defining the state vector and its dynamic model, the Kalman Filter is able under certain conditions to provide optimal filtering for measurements and their corresponding covariance matrices. Under the setting of 6-DOF tracking, the Kalman Filter described above can be fed with full (r, q) measurements provided directly from electromagnetic tracking system (EMTS) 10, alongside with their corresponding C(r,q) 7×7 covariance matrices. (Converting between C_{6×6}(r, Θ) to C_{7×7}(r, q) is straightforward using standard Euler angles to quaternion conversion formulas).

It should be noted that the local tracking error does not include static error for example due to uncompensated electromagnetic metal distortion (the compensation of which is out of the scope of the present disclosure). It only captures the local solver noise of an optimal solver at a certain position and orientation, in a neutral (distortion-free) electromagnetic environment.

Since the local tracking error increases proportionally with the sensor error and decreases proportionally with the magnetic field strength, it is convenient to assume a fixed electrical transmission current (for example 1 Ampere) and a fixed sensor noise (for example 1 uT standard deviation). By minimizing the overall tracking error for a given current and sensor noise, the optimal coils geometry is found as to decrease the overall noise of the final solved position and orientation in the sensing region of interest. Since both the sensor noise and the electrical transmission current are now fixed, as well as rₖ which are only chosen once, local tracking error estimation Cₜₒₜ is only a function of for example, crossover coils 21 and a non-crossover coil 20 geometry. Each i-th coil geometry can be described as a set of line segments between 3-dimensional vertices [pᵢⱼ, p_{i,j+1}]. Local tracking error estimation Cₜₒₜ can then be though of as a function of pᵢⱼ:

$${C}_{tot} = {C}_{tot} ( {p}_{ij} )$$

The optimal coils geometry is then found by minimizing Cₜₒₜ(pᵢⱼ) using global and/or local optimization methods such as Gradient-Descent, Newton-Raphson, Nelder-Mead, Trust Region, Levenberg-Marquardt, Genetic Algorithms, Random Search, Brute-force search methods (grid) and more. The optimal coils geometry can be searched globally for example using multiple random seed geometries, or can converge locally from a user specified initial configuration. For example, A user may specify a certain desired configuration which can be refined locally during optimization by minimizing Cₜₒₜ, starting at a specified configuration of a user.

Since the number of vertices may be rather large (in case for example of PCB coils consisting of 100 rectangular windings), optimizing over local tracking error estimation Cₜₒₜ(pᵢⱼ) may be slow. It is therefore desirable to reduce the number of parameters. For example, in the case of a planar PCB transmitter, each coil can be assumed to lie on a specific PCB layer with a specific z value. This means that the z component of each coil vertex pᵢⱼ is constant and can be removed from the optimization.

In addition, as discussed above, each coil can be very accurately approximated using just a few numbers of fundamental loops. For the sake of optimization, each coil can even be approximated using just a single fundamental loop, which for example reduces the number of vertices of a 100 rectangular windings PCB coil from 400 to just 4. The Jacobian of Cₜₒₜ(pᵢⱼ) becomes then a function of a very few parameters which can be computed very efficiently using methods discussed above (using efficient field and Jacobian computations with vector instructions). This allows for practical optimization over Cₜₒₜ(pᵢⱼ) in cases of various system configurations (3-coil planar transmitter with 3-axis receiver, 8-coil planar transmitter with 1-axis receiver etc.) for very complex coil geometries regardless of the number of windings (approximating using fundamental loops). With just a small number of parameters, the Jacobian of Cₜₒₜ(pᵢⱼ) may be estimated using numerical methods to speed up the optimization process.

Instead of assuming a fixed transmission current, it may be more desirable to assume a fixed voltage. This can be achieved as follows: for each coil geometry pᵢⱼ the exact coil resistance can be calculated using the methods described above with respect to self-crossing coil configurations 70, 80, and 90. The transmission current is then computed using Ohm's law, and it is just a function of the coil geometry: I=I(pᵢⱼ). I(pᵢⱼ) is then plugged into Cₜₒₜ(pᵢⱼ) (affecting the Jacobians) so that Cₜₒₜ(pᵢⱼ) remains a function of nothing but the coils geometry pᵢⱼ. Alternatively, one may wish to find the optimal coils for a certain fixed power dissipation, which can be described using similar methods or by posing optimization constraints (for example, punishing geometries which yield estimated power dissipation greater than the limit). Additional constraints may be added to the optimization, for example-confining the geometry to a certain region (useful for a PCB of limited size), posing a limit on length of each coil, etc. The optimization framework is extremely general and is able to combine the very accurate tracking system error estimate Cₜₒₜ with any additional constraint.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The terms “position”, “orientation” or “location” are used herein interchangeably and can mean the 3-dimensional (3D) position and/or 2 or 3-angles orientation of a receiver in a 5-degrees of freedom (DOF) or 6-DOF tracking scheme.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-21. (canceled)

22. A transmitter coil for position-finding, the transmitter coil comprising:
a plurality of turns of a conductor comprising traces of a printed circuit board, and comprising a first region of clockwise turns and a second region of counterclockwise turns configured to reduce mutual inductance between the first and second regions;
a connection that receives alternating electric current to cause the transmitter coil to transmit a total electromagnetic field into a sensing region, the total electromagnetic field being measurable within the sensing region to track a sensor;
wherein the turns are constrained by widths of the traces to converge toward a center, resulting in differing resulting magnetic fields from each turn.

23. The transmitter coil of claim 22, wherein:
a first method of calculating the total electromagnetic field accounts individually for each of the plurality of turns;
a second method of calculating the total electromagnetic field accounts for the plurality of turns as a lesser number of fundamental loops, each fundamental loop providing a portion of the total electromagnetic field; and
convergence of the traces toward the center is sufficiently great that total electromagnetic field calculated for the second method approaches total electromagnetic field calculated for the first method to within about 0.1% inside the sensing region only when at least three of said fundamental loops are used.

24. The transmitter coil of claim 23, wherein each of said fundamental loops comprises a plurality of fundamental segments, each fundamental segment having a virtual current.

25. The transmitter coil of claim 22, wherein, for a same trace width, same transmitter coil overall dimensions, same level of power dissipation, and same sensor configuration, overall tracking error in position measurements made using the transmitter coil is at least 30% reduced compared to a printed circuit board coil without a reversing direction of turn.

26. The transmitter coil of claim 22, wherein the turns of the conductor generate reversal of winding direction from clockwise to counterclockwise by crossing over each other.

27. The transmitter coil of claim 22, provided on the printed circuit board with at least two other transmitter coils.

28. The transmitter coil of claim 27, wherein at least one of the other transmitter coils does not reverse winding direction between clockwise and counterclockwise.

29. The transmitter coil of claim 23, provided together with a processor and memory storing instructions, wherein the instructions instruct the processor to:
access measurements of the total electromagnetic field in the sensing region; and
convert the accessed measurements into estimated positions relative to the transmitter coil.

30. The transmitter coil of claim 29, wherein each fundamental loop is associated with an analytic expression for its respective portion of the total electromagnetic field determined by the second method of calculating, and the analytic expressions are used by the processor to convert the accessed measurements to position estimates.

31. The transmitter coil of claim 30, wherein the processor converts the accessed measurements to position estimates, using a combination of analytic expressions determined for at least three fundamental loops.

32. The transmitter coil of claim 29, wherein the processor estimates positions with at least five degrees of freedom.

33. The transmitter coil of claim 22, provided together with a DC magnetometer configured to measure the total electromagnetic field for the position-finding, the DC magnetometer having a sensitivity substantially unaffected by frequency of the total electromagnetic field.

34. The transmitter coil of claim 22, wherein the first and second regions are adjacently positioned.

35. The transmitter coil of claim 22, wherein the first and second regions are overlappingly positioned.

36. A method of operating a transmitter coil for position measurement, the method comprising:
providing a transmitter coil comprising a plurality of turns of a conductor comprising traces of a printed circuit board, the turns comprising a first region of clockwise turns and a second region of counterclockwise turns configured to reduce mutual inductance;
providing alternating electric current to the transmitter coil to transmit a total electromagnetic field into a sensing region;
accessing measurements of the total electromagnetic field made using a sensor within the sensing region; and
calculating a position of the sensor, according to the accessed measurements.

37. The method of claim 36, performed in a medical setting with a metal frame of a patient support surrounding the transmitter coil.

38. The method of claim 36, wherein:
the calculating accounts for the plurality of turns as a plurality of fundamental loops;
within the sensing region, the calculating using the plurality of fundamental loops uses a magnetic field estimation which is within about 0.1% of magnetic fields calculated by accounting individually for electromagnetic field contributions of each turn; and
the plurality of fundamental loops is selected to compensate for differences in resulting magnetic fields produced by the traces as they converge toward a center, such that obtaining the error within about 0.1% of the result requires the calculating to use at least three fundamental loops.

39. The method of claim 38, wherein the calculating converts the accessed measurements to position estimates using a combination of analytic expressions determined for at least three fundamental loops.

40. A method of designing a transmitter coil manufactured as part of a printed circuit board, the method comprising:
selecting power constraints for the transmitter coil, and dimensional constraints of the printed circuit board;
selecting an initial number of transmitter coil turns, sized to fit within the dimensional constraints of the printed circuit board, while maintaining material construction and cross-sectional size consistent with the power constraints; and
iteratively adjusting geometries of the transmitter coil turns within the power constraints and the dimensional constraints;
wherein the adjusting geometries is performed to optimize at least one of the group consisting of calculated overall tracking error and calculated power dissipation.

41. The method of claim 40, wherein:
calculations to evaluate the iteratively adjusted geometries of the transmitter coil turns use an approximation which groups the transmitter coil turns into a plurality of fundamental loops;
the plurality of fundamental loops comprises at least three fundamental loops;
for a volume of a sensing region in which position relative to the transmitter coil is to be determined during use of the transmitter coil, the approximation using fundamental loops allows position estimation based on measurements of an electromagnetic field produced by the transmitter coil, with an error of approximation for the electromagnetic field within about 0.1% of electromagnetic field calculation considering each individual transmitter coil turn;
wherein said error of approximation is reached only when at least three of said fundamental loops are used.

42. The method of claim 41, wherein:
convergence of the turns toward a center of the transmitter coil has an effect on resulting magnetic fields sufficiently significant so as to constrain the calculating to use at least three of said fundamental loops; and
wherein an efficiency gained from optimization of the transmitter coil designs is at least a 30% decrease in overall tracking error for a same power level, or at least a 30% reduction in power level for a same overall tracking error.
